(19) United States
(12) Patent Application Publication
(10) Pub. No.: US 2025/0354196 A1
(43) Pub. Date: Nov. 20, 2025

(54) BORANES ON SOLID SUPPORTS

(71) Applicant: Illumina, Inc., San Diego, CA (US)

(72) Inventors: Carole Anastasi, Cambridge (GB); Elena Cressina, Cambridge (GB); Xiaohai Liu, Cambridge (GB); Xavier von Hatten, Cambridge (GB); Gianluca Artioli, Cambridge (GB); Teodora Gal, Cambridge (GB)

(73) Assignee: Illumina, Inc., San Diego, CA (US)

(21) Appl. No.: 18/867,398

(22) PCT Filed: Dec. 14, 2023
(86) PCT No.: PCT/US2023/084128

§ 371 Date: Nov. 19, 2024

Related U.S. Application Data
(60) Provisional application No. 63/387,906, filed on Dec. 16, 2022.

(51) Int. Cl. C12Q 1/6806; C12Q 1/26

(52) U.S. Cl. CPC C12Q 1/6806; C12Q 1/26; C12Y 114/11; C12Q 2600/154

(57) ABSTRACT

Disclosed herein are methods and compositions that utilize boranes on solid supports. The methods and compositions can be used to detect methylation on polynucleotides. In some examples, a method includes oxidizing any 5-methylcytosine (5-mC) or 5-hydroxymethylcytosine (5-hmC) on a polynucleotide to 5-carboxylcytosine (5-caC) or 5-formylcytosine (5-fC). The method may include reducing the 5-caC or 5-fC to 5,6-dihydrouracil (DHU) using an amine-borane attached to a solid support. The method may include detecting the 5-methylcytosine or 5-hydroxymethylcytosine using the DHU.

### CROSS-REFERENCE TO RELATED APPLICATIONS

This application relates to borane compositions, such as may be used to detect methylated cytosines.

### INCORPORATION BY REFERENCE OF SEQUENCE LISTING

The material in the accompanying sequence listing is hereby incorporated by reference into the application. The accompanying sequence listing XML file, named “85491_05716.xml”, was created on Dec. 14, 2023 and is 12 kB in size.

### FIELD

This application relates to borane compositions, such as may be used to detect methylated cytosines.

### BACKGROUND

Within living organisms, such as humans, selected cytosines in the genome may become methylated. Methods to detect methylated cytosines include using sodium bisulfite and borane-containing compounds. However, a major issue with these methods is that significant amounts of DNA are often degraded. Thus, new methods and compositions are needed to detect methylated DNA that are less toxic to DNA than the methods and compositions currently on the market.

### SUMMARY

Some examples herein provide a method including oxidizing any 5-methylcytosine (5-mC) or 5-hydroxymethylcytosine (5-hmC) on a polynucleotide to 5-carboxylcytosine (5-caC) or 5-formylcytosine (5-fC); reducing the 5-caC or 5-fC to 5,6-dihydrouracil (DHU) using an amine-borane attached to a solid support; and detecting the 5-methylcytosine or 5-hydroxymethylcytosine using the DHU.

In some examples, the solid support includes any one or more of a bead, a microsphere, a filter, a surface of a tube or vessel, and a planar substrate.

In some examples, the bead includes a magnetic bead. In some examples, the bead includes a paramagnetic bead. In some examples, the microsphere includes a magnetic microsphere.

In some examples, the bead includes a solid-phase reversible immobilization (SPRI) bead.

In some examples, ten-eleven translocation (TET) dioxygenase is used to oxidize any 5-mC or 5-hmC. In some examples, oxidizing any 5-mC or 5-hmC includes contacting 5-mC or 5-hmC with one or more chemical reagents.

In some examples, the method further includes contacting the solid support with the 5-caC or 5-fC. In some examples, the solid support includes a magnetic bead.

In some examples, the method further includes using the solid support to separate the DHU from the amine-borane and any other reaction products. In some examples, the solid support includes a magnetic bead.

In some examples, the oxidizing step and reducing step take place in a solution. In some examples, the method further includes absorbing the polynucleotide onto the solid support. In some examples, the method further includes eluting the polynucleotide such that it is released from the solid support. In some examples, the polynucleotide remains in solution.

In some examples, the solid support is attached to a linker and the amine-borane attaches to the solid support through the linker.

Some examples herein provide a composition that includes a magnetic bead coupled to an amine-borane.

In some examples, the composition further includes a linker that couples the magnetic bead to the amine-borane.

In some examples, the magnetic bead comprises a SPRI bead. In some examples, the magnetic bead comprises a paramagnetic bead.

In some examples, the magnetic bead is connected to a first functional group (F₁) and the linker is connected to a second functional group (F₂) and to a third functional group (F₃), and the linker couples the magnetic bead to the amine-borane via coupling F₁ to F₂, and coupling F₃ to the amine-borane.

Some examples herein provide a method that includes coupling a magnetic bead to a linker to create a composite magnetic bead-linker structure; and coupling the composite magnetic bead-linker structure to an amine-borane.

In some examples, the magnetic bead is connected to a first functional group (F₁) and the linker is connected to a second functional group (F₂) and to a third functional group (F₃), and the magnetic bead is coupled to the linker via coupling F₁ to F₂ to form the composite magnetic bead-linker structure, and the composite magnetic bead-linker structure is coupled to the amine-borane via coupling F₃ to the amine-borane.

It is to be understood that any respective features/examples of each of the aspects of the disclosure as described herein may be implemented together in any appropriate combination, and that any features/examples from any one or more of these aspects may be implemented together with any of the features of the other aspect(s) as described herein in any appropriate combination to achieve the benefits as described herein.

### BRIEF DESCRIPTION OF THE DRAWINGS

FIG. 1 schematically illustrates an example workflow for detecting methylation using a borane that is attached to a solid support such as a bead.

FIG. 2A schematically illustrates an example of a bead that is attached to an amine-borane using a linker.

FIGS. 2B-2C schematically illustrate examples of linkers. FIG. 2B schematically illustrates examples of branched linkers. FIG. 2C schematically illustrates examples of dendritic linkers.

FIGS. 3A-3B schematically illustrate example operations for coating functionalized magnetic beads with a functionalized hydrogel to which a borane subsequently may be coupled. FIG. 3A schematically illustrates an example operation for coating beads with an azido-functionalized hydrogel. FIG. 3B schematically illustrates an example of converting azido groups of the hydrogel into amines to which a borane may subsequently be coupled.

FIG. 4 schematically illustrates examples of coupling a bead to an amine-borane using a linker.

FIGS. 5A-5E schematically illustrate examples of possible coupling reactions in which beads are attached to amine-boranes.

FIGS. 6A-6C schematically illustrate examples of class B boranes attached to beads.

FIGS. 6D-6F schematically illustrate examples of class A boranes attached to beads.

FIGS. 7A-7B illustrate data showing that class B boranes can slowly convert caCpG into DHUpG, on dinucleotides.

FIGS. 8A-8B illustrate data showing that class A boranes are more effective than class B boranes in converting caCpG into DHUpG, on dinucleotides.

FIGS. 9A-9B illustrate data showing that class A boranes are effective in converting caCpG to DHUpG, on 7-mer oligonucleotides (5′-ATcaCGCTA-3′). FIG. 9C illustrates an example embodiment of a borane construct attached to a bead that was used in the experiments that produced the data in FIGS. 9A and 9B.

FIGS. 10A-10B and 10D illustrate data showing that class A boranes are effective in converting caCpG to DHUpG, on 20-mer oligonucleotides (5′-TTTCAGCTCcaCGGTCACGCTC-3′) (SEQ ID NO: 1). FIG. 10C illustrates an example embodiment of a borane construct attached to a bead that was used in the experiments that produced the data shown in FIGS. 10A-10B and 10D.

### DETAILED DESCRIPTION

Examples provided herein are related to methods and compositions in which amine-boranes are attached to solid supports. In some examples, the solid supports include beads. In some examples, the methods and compositions are used to detect methylated cytosines on polynucleotides.

For example, as provided herein, 5-methylcytosine (5-mC) or 5-hydroxymethylcytosine (5-hmC) on a polynucleotide can be oxidized to form 5-carboxylcytosine (5-caC) or 5-formylcytosine (5-fC). The 5-caC or 5-fC can be reduced to 5,6-dihyrouracil (DHU) using an amine-borane that is attached to a solid support. The 5-mC or 5-hmC can be detected using the DHU. The oxidizing step can first take place in a solution and then the reduction can take place on a solid support. In some examples, the solid support can be a bead or a magnetic bead. In some examples, the bead or magnetic bead is connected to the amine-borane via a linker.

First, some terms used herein will be briefly explained. Then, some example compositions and example methods using the compositions will be described.

### Terms

Unless defined otherwise, all technical and scientific terms used herein have the same meaning as is commonly understood by one of ordinary skill in the art. The use of the term “including” as well as other forms, such as “include,” “includes,” and “included,” is not limiting. The use of the term “having” as well as other forms, such as “have,” “has,” and “had,” is not limiting. As used in this specification, whether in a transitional phrase or in the body of the claim, the terms “comprise(s)” and “comprising” are to be interpreted as having an open-ended meaning. That is, the above terms are to be interpreted synonymously with the phrases “having at least” or “including at least.” For example, when used in the context of a process, the term “comprising” means that the process includes at least the recited steps, but may include additional steps. When used in the context of a compound, composition, or device, the term “comprising” means that the compound, composition, or device includes at least the recited features or components, but may also include additional features or components.

The terms “substantially,” “approximately,” and “about” used throughout this specification are used to describe and account for small fluctuations, such as due to variations in processing. For example, they may refer to less than or equal to ±10%, such as less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to ±0.05%.

As used herein, the term “nucleotide” is intended to mean a molecule that includes a sugar and at least one phosphate group, and in some examples also includes a nucleobase. A nucleotide that lacks a nucleobase may be referred to as “abasic.” Nucleotides include deoxyribonucleotides, modified deoxyribonucleotides, ribonucleotides, modified ribonucleotides, peptide nucleotides, modified peptide nucleotides, modified phosphate sugar backbone nucleotides, and mixtures thereof. Examples of nucleotides include adenosine monophosphate (AMP), adenosine diphosphate (ADP), adenosine triphosphate (ATP), thymidine monophosphate (TMP), thymidine diphosphate (TDP), thymidine triphosphate (TTP), cytidine monophosphate (CMP), cytidine diphosphate (CDP), cytidine triphosphate (CTP), guanosine monophosphate (GMP), guanosine diphosphate (GDP), guanosine triphosphate (GTP), uridine monophosphate (UMP), uridine diphosphate (UDP), uridine triphosphate (UTP), deoxyadenosine monophosphate (dAMP), deoxyadenosine diphosphate (dADP), deoxyadenosine triphosphate (dATP), deoxythymidine monophosphate (dTMP), deoxythymidine diphosphate (dTDP), deoxythymidine triphosphate (dTTP), deoxycytidine diphosphate (dCDP), deoxycytidine triphosphate (dCTP), deoxyguanosine monophosphate (dGMP), deoxyguanosine diphosphate (dGDP), deoxyguanosine triphosphate (dGTP), deoxyuridine monophosphate (dUMP), deoxyuridine diphosphate (dUDP), and deoxyuridine triphosphate (dUTP).

As used herein, the term “nucleotide” also is intended to encompass any nucleotide analogue which is a type of nucleotide that includes a modified nucleobase, sugar and/or phosphate moiety compared to naturally occurring nucleotides. Example modified nucleobases include inosine, xanthine, hypoxanthine, isocytosine, isoguanine, 2-aminopurine, 5-methylcytosine, 5-hydroxymethyl cytosine, 2-aminoadenine, 6-methyl adenine, 6-methyl guanine, 2-propyl guanine, 2-propyl adenine, 2-thiouracil, 2-thiothymine, 2-thiocytosine, 5-halouracil, 5-halocytosine, 5-propynyl uracil, 5-propynyl cytosine, 6-azo uracil, 6-azo cytosine, 6-azo thymine, 5-uracil, 4-thiouracil, 8-halo adenine or guanine, 8-amino adenine or guanine, 8-thiol adenine or guanine, 8-thioalkyl adenine or guanine, 8-hydroxyl adenine or guanine, 5-halo substituted uracil or cytosine, 7-methylguanine, 7-methyladenine, 8-azaguanine, 8-azaadenine, 7-deazaguanine, 7-deazaadenine, 3-deazaguanine, 3-deazaadenine or the like. As is known in the art, certain nucleotide analogues cannot become incorporated into a polynucleotide, for example, nucleotide analogues such as adenosine 5′-phosphosulfate. Nucleotides may include any suitable number of phosphates, e.g., three, four, five, six, or more than six phosphates.

As used herein, the term “polynucleotide” refers to a molecule that includes a sequence of nucleotides that are bonded to one another. A polynucleotide is one nonlimiting example of a polymer. Examples of polynucleotides include deoxyribonucleic acid (DNA), ribonucleic acid (RNA), and analogues thereof. A polynucleotide may be a single stranded sequence of nucleotides, such as RNA or single stranded DNA, a double stranded sequence of nucleotides, such as double stranded DNA, or may include a mixture of a single stranded and double stranded sequences of nucleotides. Double stranded DNA (dsDNA) includes genomic DNA, and PCR and amplification products. Single stranded DNA (ssDNA) can be converted to dsDNA and vice-versa. Polynucleotides may include non-naturally occurring DNA, such as enantiomeric DNA. The precise sequence of nucleotides in a polynucleotide may be known or unknown. The following are examples of polynucleotides: a gene or gene fragment (for example, a probe, primer, expressed sequence tag (EST) or serial analysis of gene expression (SAGE) tag), genomic DNA, genomic DNA fragment, exon, intron, messenger RNA (mRNA), transfer RNA, ribosomal RNA, ribozyme, cDNA, recombinant polynucleotide, synthetic polynucleotide, branched polynucleotide, plasmid, vector, isolated DNA of any sequence, isolated RNA of any sequence, nucleic acid probe, primer or amplified copy of any of the foregoing.

The terms “polynucleotide” and “oligonucleotide” are used interchangeably herein. The different terms are not intended to denote any particular difference in size, sequence, or other property unless specifically indicated otherwise. For clarity of description the terms may be used to distinguish one species of polynucleotide from another when describing a particular method or composition that includes several polynucleotide species.

As used herein, the term “methylcytosine” or “mC” refers to cytosine that includes a methyl group (—CH₃ or -Me). The methyl group may be located at the 5 position of the cytosine, in which case the mC may be referred to as 5mC or 5-mC.

As used herein, a “derivative” of methylcytosine refers to methylcytosine having an oxidized methyl group. A nonlimiting example of an oxidized methyl group is hydroxymethyl (—CH₂OH), in which case the mC derivative may be referred to as hydroxymethylcytosine or hmC. Another nonlimiting example of an oxidized methyl group is formyl group (—CHO) in which case the mC derivative may be referred to as formylcytosine or fC. Another nonlimiting example of an oxidized methyl group is carboxyl (—COOH), in which case the mC derivative may be referred to as carboxylcytosine or caC. The oxidized methyl group may be located at the 5 position of the cytosine, in which case the hmC may be referred to as 5hmC or 5-hmC, the fC may be referred to as 5fC or 5-fC, or the caC may be referred to as 5caC or 5-caC. The fC optionally may be present in an acetal form (—CH(OH)₂). The caC optionally may be present in a salt form (—COO).

As used herein, the terms “amine-borane complex” refers to a chemical compound that includes a borane which is bonded to a nitrogen within a heterocyclic organic molecule. The heterocyclic organic molecule optionally may include one or more additional heterocyclic atoms besides the nitrogen which is bonded to the borane. In various examples, the heterocyclic organic molecule may include a substituted pyridine, an azole, a pyrimidine, or a pyrazine. As such, the amine-borane complex may include a substituted pyridine borane complex, an azole borane complex, or a pyrimidine borane complex. The terms “amine-borane complex” are used interchangeably with the terms “amine-borane.”

As used herein, the term “PAZAM” refers to the hydrogel polymer: poly(N-(5-azidoacetamidylpentyl) acrylamide-co-acrylamide). A PAZAM bead refers to a bead that is coated with the hydrogel polymer: poly(N-(5-azidoacetamidylpentyl) acrylamide-co-acrylamide).

As used herein, the term “SPRI” is synonymous with the phrase: solid phase reversible immobilization. SPRI beads are magnetic beads or paramagnetic beads that are functionalized on their surface with carboxylic acid groups. These carboxylic acid functions can be subsequently used to anchor other chemical compounds such as amine borane compounds.

As used herein, the phrases “Class A Boranes” and “Class B Boranes” are relative phrases that describe relative rates that the classes of boranes convert caCpG to DHUpG. A “Class A Borane” converts caCpG to DHUpG at a faster rate than pyridine borane converts caCpG to DHUpG. In some examples, a “Class A Borane” may convert caCpG to DHUpG at a rate that is 2× or 3× faster than pyridine borane converts caCpG to DHUpG. In some examples, a “Class A Borane” may convert caCpG to DHUpG at a rate that is more than 3× faster than pyridine borane converts caCpG to DHUpG. A “Class B Borane” converts caCpG to DHUpG at a rate similar to the rate that pyridine borane converts caCpG to DHUpG.

### Methods of Detecting Methylated Polynucleotides Using Amine-Boranes on Solid Supports

Some examples provided herein relate to a method that includes oxidizing any 5-methylcytosine (5-mC) or 5-hydroxymethylcytosine (5-hmC) in a polynucleotide to 5-carboxylcytosine (5-caC) or 5-formylcytosine (5-fC); reducing the 5-caC or 5-fC to 5,6-dihydrouracil (DHU) using an amine-borane attached to a solid support; and detecting the 5-methylcytosine or 5-hydroxymethylcytosine using the DHU.

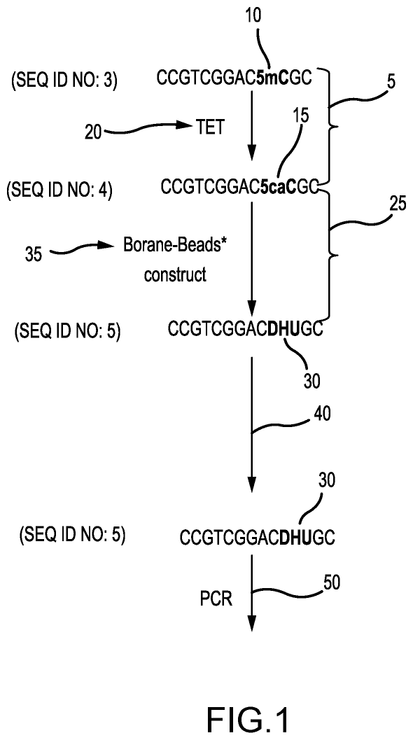

FIG. 1 schematically illustrates an example workflow for detecting methylation using a borane that is attached to a solid support such as a bead. The workflow shown in FIG. 1 includes oxidizing any 5-methylcytosine (5-mC) or 5-hydroxymethylcytosine (5-hmC) in a polynucleotide to 5-carboxylcytosine (5-caC) or 5-formylcytosine (5-fC). For example, as shown in FIG. 1, the oxidizing step (5) converts 5-mc (10) to a 5-caC (15). Optionally, additionally or alternatively, the oxidation step can convert 5-hmC to 5-fC. In some examples, ten-eleven translocation (TET) dioxygenase (20) can be used as the oxidizing agent. Optionally, other oxidizing agents described herein can be used. In some nonlimiting examples using one or more chemical reagents, 5-mC may be oxidized to 5-caC using menadione, ultraviolet (UV) radiation at 365 nm, under oxygen, followed by 2,2,6,6-tetramethyl-1-piperidinyloxy free radical (TEMPO)/bis(acetoxyiodobenzene) (BAIB) in a manner such as described in Kore et al., “Concise synthesis of 5-methyl, 5-formyl, and 5-carboxy analogues of 2′-deoxycytidine-5′-triphosphate,” Tetrahedron letters 54 (39): 5325-5327 (2013), the entire contents of which are incorporated by reference herein. In other nonlimiting examples using one or more chemical reagents, 5-hmC or 5-fC may be oxidized to 5-caC using TEMPO/BAIB in a manner such as described in Sun et al., “Efficient synthesis of 5-hydroxymethyl-, 5-formyl-, and 5-carboxyl-2′-deoxycytidine and their triphosphates,” RSC Advances 4 (68): 36036-36039 (2014), the entire contents of which are incorporated by reference herein. In still other nonlimiting examples using one or more chemical reagents, an iron (IV)-oxo complex is used to oxidize 5-mC to 5-caC in a manner such as described in Schmidl et al., “Biomimetic iron complex achieves TET enzyme reactivity,” Angewandte Chemie Int'l Ed. 60 (39): 21457-21463 (2021), the entire contents of which are incorporated by reference herein.

The workflow shown in FIG. 1 also includes reducing the 5-caC or 5-fC to 5,6-dihydrouracil (DHU) using an amine-borane attached to a solid support. For example, in FIG. 1, the oxidizing step (5) is followed by a reducing step (25) in which the 5-caC (15) is converted to DHU (30). An amine-borane attached to a bead (35) can be used in the reduction step. In some examples, the bead includes a solid-phase reversible immobilization (SPRI) bead. In some examples, the SPRI bead is or includes any SPRI bead described herein. In some examples, the bead includes a PAZAM bead. In some examples, the PAZAM bead includes any PAZAM described herein.

Alternatively, the amine-borane can be attached to other solid supports described herein such as any one or more of a microsphere, a filter, a surface of a tube or vessel, and a planar substrate. In some examples, the solid support includes an inert substrate or matrix, such as, for example, glass beads or polymer beads. In some examples, the amine-borane is attached to the solid support through a covalent linkage between the amine-borane and the solid support. In some examples, the solid support is magnetic. In some examples, the solid support is paramagnetic.

In some examples, the amine-borane is immobilized on the solid support.

In some examples, the method further includes using the solid support to separate the DHU from the amine-borane and any other reaction products.

As shown in FIG. 1, following the reduction step (25), the polynucleotide may be coupled to (e.g., absorbed on) the solid support (e.g., beads) or remain in solution (40). In examples in which the polynucleotide is coupled to (e.g., absorbed on) the solid support, the polynucleotide may then be washed and eluted. In examples in which the polynucleotide (e.g., DNA) remains in solution, the polynucleotide may be separated from the solid support and purified using standard techniques known in the art.

In some examples, the oxidizing step and reducing step take place in a solution.

In the nonlimiting example illustrated in FIG. 1, a pH of 4.3 can be used in the reduction step (25). Any alternative pH described herein can also be used in the reduction step. In some examples, a pH between 3.7 and 4.9 is used in the reduction step, for example, a pH of approximately 3.7, a pH of approximately 3.8, a pH of approximately 3.9, a pH of approximately 4.0, a pH of approximately 4.1, a pH of approximately 4.2, a pH of approximately 4.3, a pH of approximately 4.4, a pH of approximately 4.5, a pH of approximately 4.6, a pH of approximately 4.7, a pH of approximately 4.8, or a pH of approximately 4.9. In some examples, a pH below 3.7 is used in the reduction step. In some examples, a pH above 4.9 is used in the reduction step.

In some examples, washing the polynucleotide includes at least one (1) wash step. In some examples, the at least one (1) wash step includes one (1) wash, two (2) washes, three (3) washes, four (4) washes, five (5) washes, or six (6) washes. In some examples, the at least one (1) wash step includes more than six (6) washes. In some examples, the at least one wash step utilizes a salt solution. In some examples, the at least one wash step utilizes an ethanol solution.

In some examples, eluting the polynucleotide includes purifying the polynucleotide. In some examples, eluting the polynucleotide includes performing chromatography, for example, ion exchange chromatography, affinity chromatography, or size-exclusion chromatography.

The workflow shown in FIG. 1 also includes detecting the 5-methylcytosine or 5-hydroxymethylcytosine using the DHU. For example, as FIG. 1 illustrates, PCR can be used to amplify the DNA (50) followed by sequencing to detect the 5-methylcytosine or 5-hydroxymethylcytosine. For example, during PCR amplification of a first sample which is processed in the manner described with reference to FIG. 1, the DHU generated through oxidizing and reducing the 5-mC or 5-hmC may be amplified as T, and thus may be sequenced as T. In comparison, any C in the first sample which is not methylated may be amplified as C, and thus may be sequenced as C. A second sample which is not processed in the manner described with reference to FIG. 1 also may be amplified using PCR. Because the 5-mC and 5-hmC in the second sample are not converted to DHU, such bases may be amplified as C, and thus may be sequenced as C. The sequences of the first sample and second sample may be compared to determine which bases were T in the first sample and C in the second sample, and such bases may be identified as being 5-mC or 5-hmC.

In some examples, the method further includes contacting the solid support with the 5-caC or 5-fC. In some examples, the solid support includes a magnetic bead. In some examples, the magnetic bead is a paramagnetic bead. In some examples, the solid support includes any solid support described herein.

In some examples, the solid support is attached to a linker and the amine-borane attaches to the solid support through the linker. In some examples, the solid support attaches to the linker using any functional group described herein.

Some examples herein provide a method, including coupling a magnetic bead to a linker to create a composite magnetic bead-linker structure; and coupling the composite magnetic bead-linker structure to an amine-borane.

Compositions that Include Amine-Boranes on Solid Supports

Some examples provided herein relate to a composition, comprising a bead coupled to an amine-borane. In some examples, the bead includes a magnetic bead. In some examples, the bead includes a paramagnetic bead.

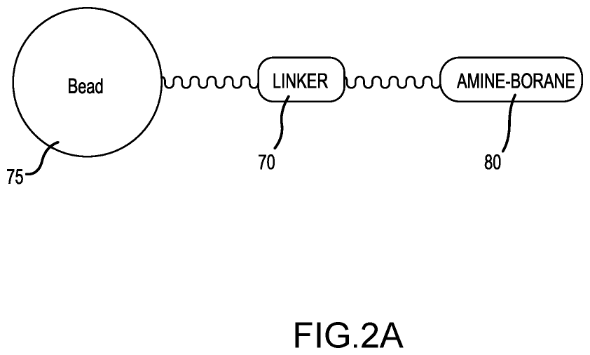

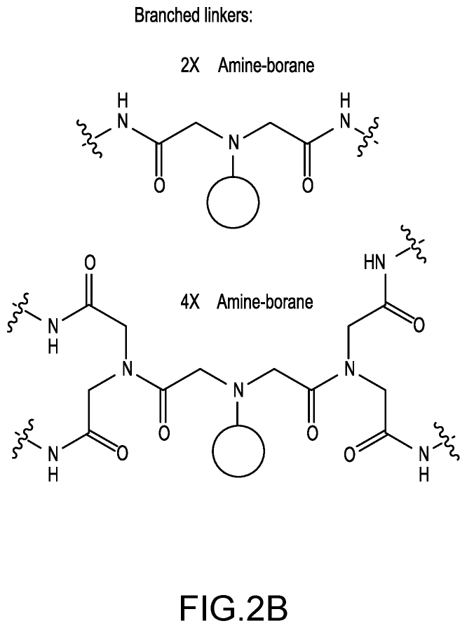

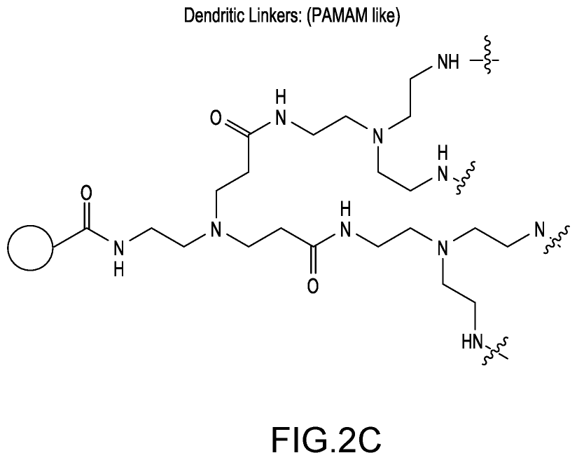

In some examples, the composition includes a linker that couples the bead to the amine-borane. In some examples, the composition includes more than one linker that couples the bead to the amine-borane. FIG. 2A schematically illustrates an example of a bead that is attached to an amine-borane using a linker. The composition in FIG. 2A includes a linker (70) that couples the bead (75) to the amine-borane (80).

In some examples, the linker includes a linear alkyl chain of any length. In some examples, the linker includes a linear polyethyleneglycol chain of any length. In some examples, the linker includes linear polyamide chains (e.g., polypeptides, aliphatic polyamides, aromatic polyamides, etc. . . . ). In some examples, the linker includes linear polyaromatic chains. In some examples, the linker includes polyamine chains. In some examples, the linkers are branched (see, for example, FIG. 2B). In some examples, the linkers are dendrimeric (see, for example, FIG. 2C).

In some examples, the linker includes any one or more of polyethylene glycol (PEG), poly(glycerol) (PG), poly(oxazoline) (POX), poly(hydroxypropyl methacrylate) (PHPMA), poly(2-hydroxyethyl methacrylate) (PHEMA), poly(N-(2-hydroxypropyl) methacrylamide) (HPMA), poly(vinylpyrrolidone) (PVP), poly(N,N-dimethyl acrylamide) (PDMA), and poly(N-acryloylmorpholine) (PAcM). In some examples, the molecular weight of the linker is between two (2) and twenty (20). In some examples, the molecular weight of the linker is greater than twenty (20).

In some examples, the linker functions to facilitate coupling of the bead to the amine-borane. In some examples, the linker increases accessibility of the bead surface, to facilitate attachment of the amine-borane to the bead. In some examples, the linker increases loading capacity of the bead, to facilitate attachment of the amine-borane to the bead, and to increase reducing capacity of the amine-borane-bead complex.

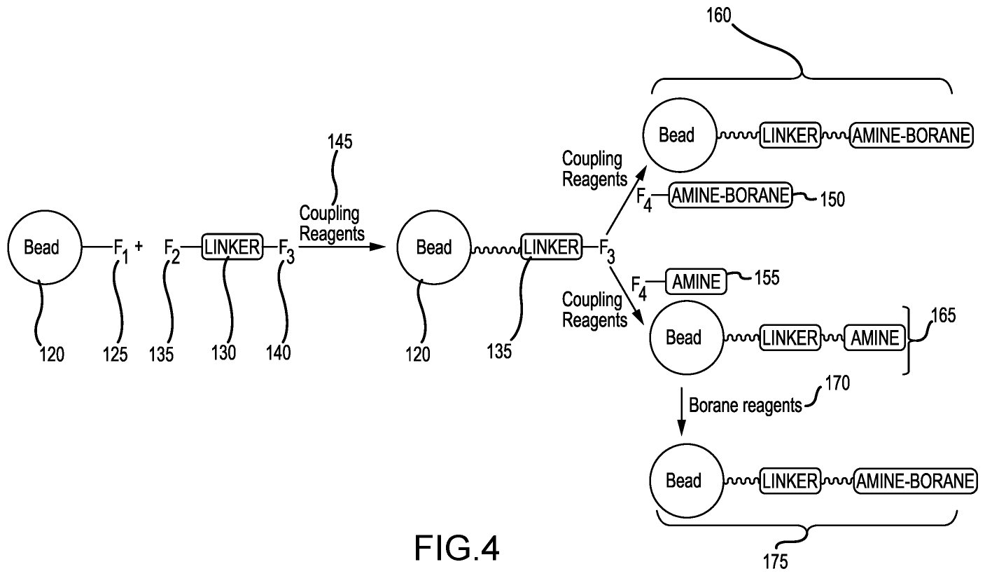

In some examples, the bead is connected to a first functional group (F₁) and the linker is connected to a second functional group (F₂) and to a third functional group (F₃), wherein the linker couples the bead to the amine-borane via coupling F₁ to F₂, and coupling F₃ to the amine-borane. FIG. 4 schematically illustrates examples of coupling a bead to an amine-borane or an amine using a linker. FIG. 4 shows an example of a bead (120) that is connected to a first functional group (F₁) (125), and a linker (130) that is connected to a second functional group (F₂) (135) and to a third functional group (F₃) (140). Using coupling reagents (145), the linker (130) couples the bead (120) to an amine-borane (150) or an amine (155) via coupling F₁ (125) to F₂ (135), and coupling F₃ (140) to F₄ of the amine-borane (150) or amine (155).

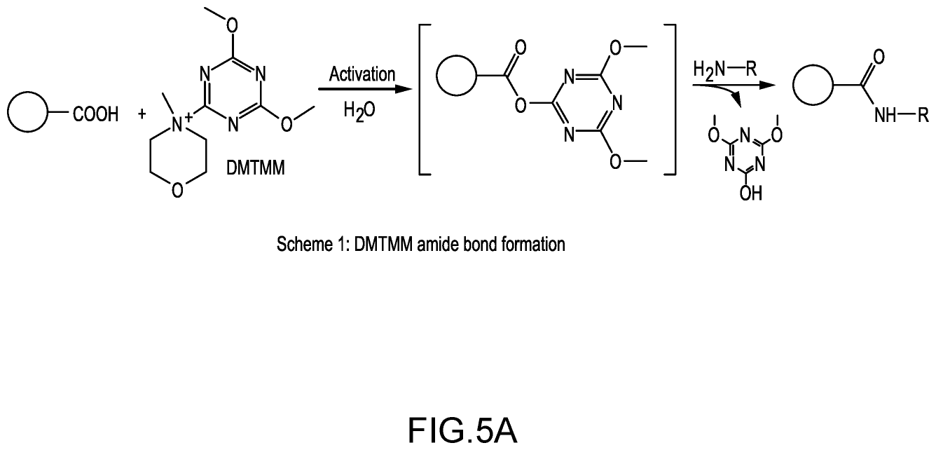

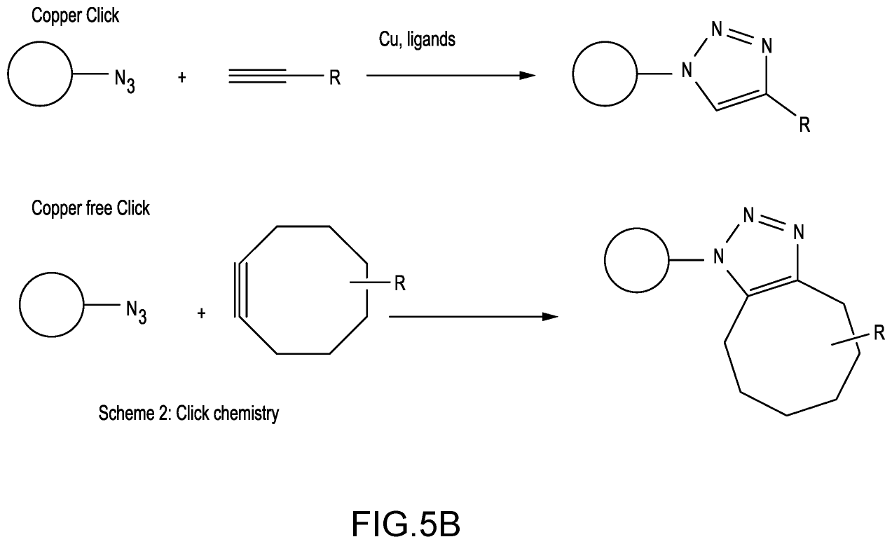

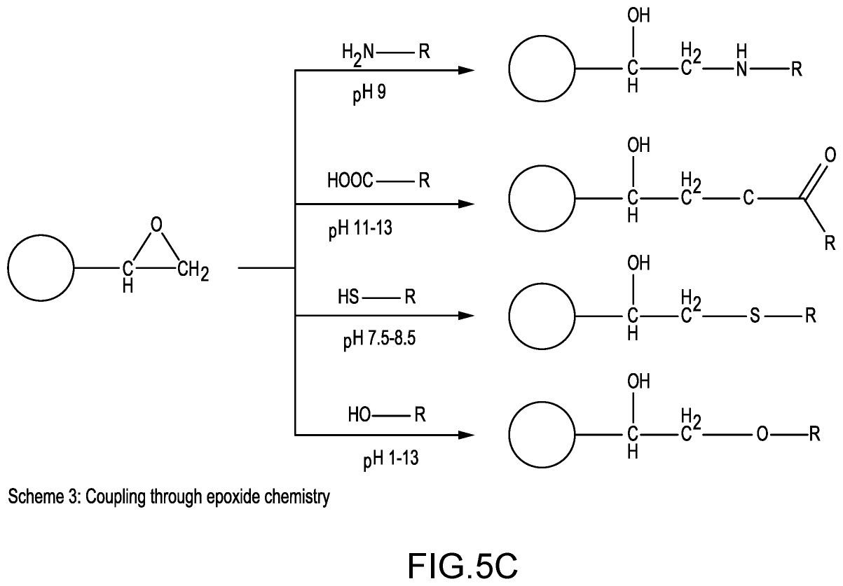

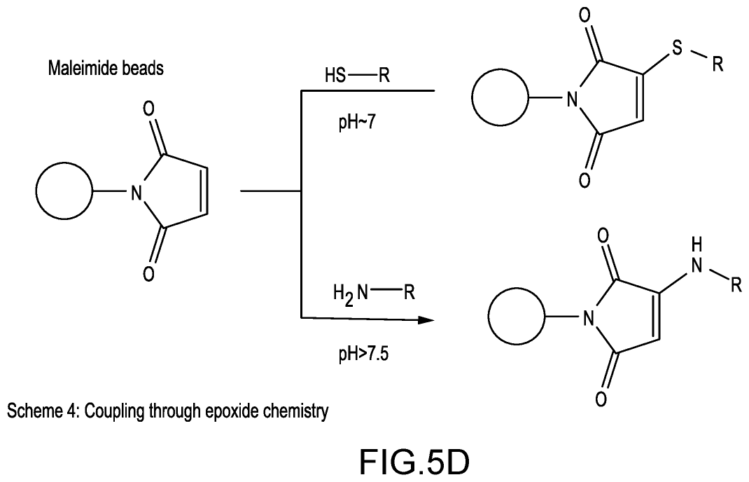

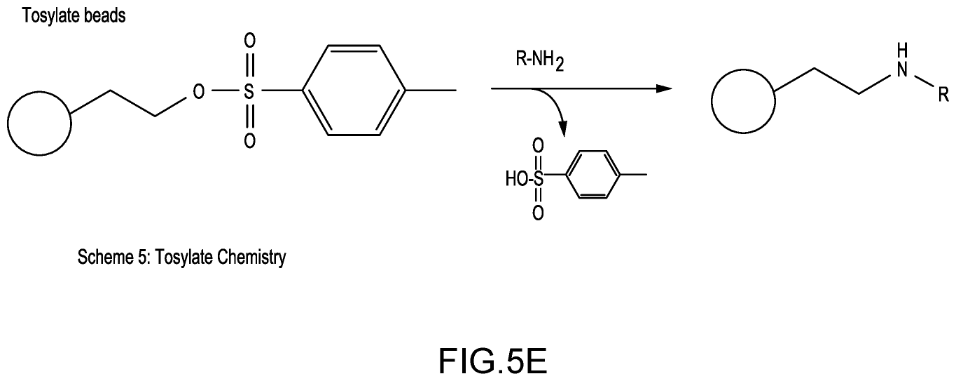

The functional groups (i.e., F₁ (125), F₂ (135), and F₃ (140)) shown in FIG. 4 can include different functional groups such as —OH, —NH₂, —COOH, —SH, and —N₃, as well as others. The coupling reagent (145) shown in FIG. 4 may allow the different functional groups to react together. The coupling reagent can be used to attach the bead to the linker, and to attach the linker to the amine (155) or the amine-borane (150). These coupling reagents can be, for example, amide coupling reagents (like DMTMM in FIG. 5A, or carbonyldiimidazole, etc. . . . ), or they can be click-chemistry reagents (any Cu(I)/ligands), etc. . . . .

As further illustrated in FIG. 4, if the linker couples the bead directly to an amine-borane, a composite structure (160) is produced that includes the bead attached to an amine-borane using a linker. Alternatively, as illustrated in FIG. 4, if the linker couples the bead to an amine, a composite structure (165) is produced that includes the bead attached to an amine using a linker. Borane reagents (170) can be used to convert the composite structure (165) to the composite structure (175) that includes a bead attached to an amine-borane using a linker. Examples of borane reagents that can be used include BH₃-THF complex, SMe₂-BH₃ complex, ammonia borane complex, NaBH₄/NaHCO₃, and 2,6-Lutidine borane.

The composite structure of (160) in FIG. 4 is the same as the composite structure of (175) in FIG. 4. This illustrates that the same composite structure can be achieved through two different schemes, as shown in FIG. 4.

In some examples, more than two (2) functional groups are used to couple the bead to the linker. In some examples, more than one (1) functional group is used to couple the linker to the amine-borane.

In some examples, the functional groups (i.e., F₁ (125), F₂ (135), and F₃ (140)) in FIG. 4 can include a hydroxyl group, an amino group, a carboxyl group, a thiol group, an azido group, or an alkyne (FIG. 5B).

In some examples, the magnetic bead is connected to a functional group and the functional group directly connects to the amine-borane, without the use of a linker.

FIGS. 5A-5E schematically illustrate examples of possible coupling reactions in which beads are attached to amine-boranes. FIG. 5A schematically illustrates a coupling reaction in which an amide bond is used (—NH₂/—COOH). Any activating agent can be used to facilitate the peptide bond formation such as DMTMM (4-(4,6-dimethoxy-1,3,5-triazin-2-yl)-4-methyl-morpholinium chloride), EDC (1-ethyl-3-(3-dimethylaminopropyl) carbodiimide hydrochloride), CDI (carbonyldiimidazole), TSTU (2-(2,5-Dioxopyrrolidin-1-yl)-1,1,3,3-tetramethylisouronium tetrafluoroborate), DCC (dicyclohexylcarbodiimide)/NHS (N-hydroxysuccinimide) as well as others. FIG. 5B schematically illustrates a coupling reaction that utilizes click chemistry coupling (—N₃/-alkyne). The reaction can proceed with or without copper. In some examples, copper is not used in the presence of borane. In some examples, a strained alkyne is used such as DBCO (Dibenzocyclooctyne). FIG. 5C schematically illustrates a coupling reaction that utilizes epoxide coupling. Epoxide can be coupled to a range of functionalities depending on the pH. FIG. 5D schematically illustrates an example of maleimide coupling. Maleimide can be coupled to amino or thiol functionalities. FIG. 5E schematically illustrates an example of tosylate coupling. Tosylate functionality can be coupled to amino groups, thiol groups, and hydroxyl groups.

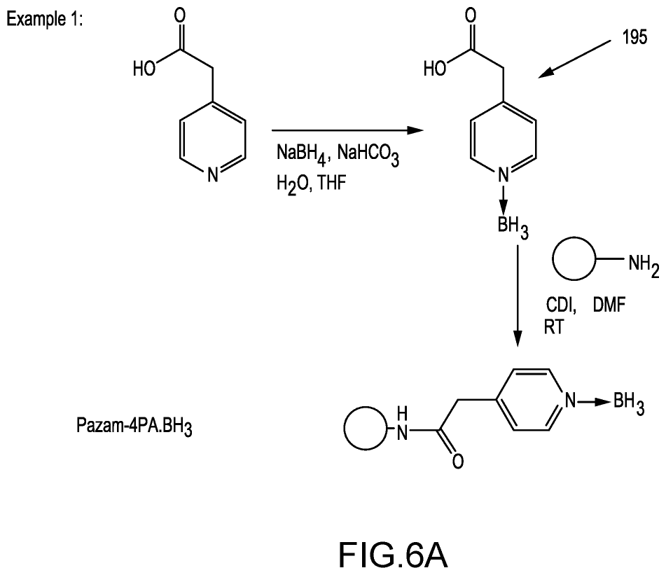

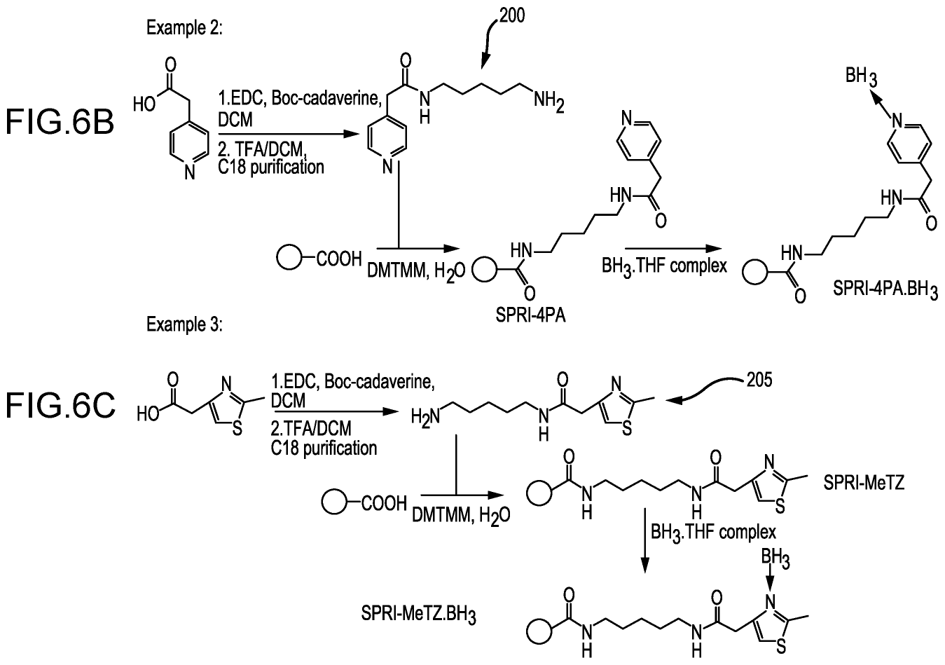

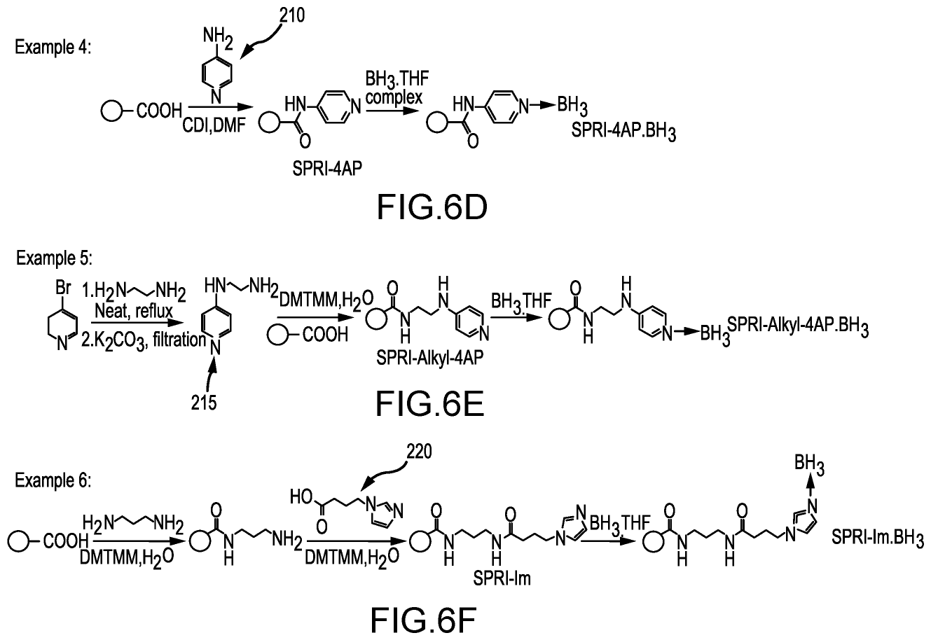

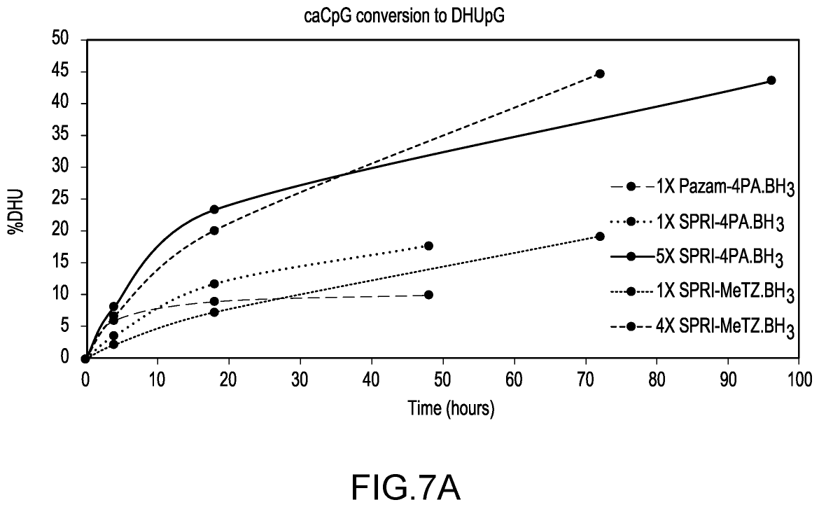

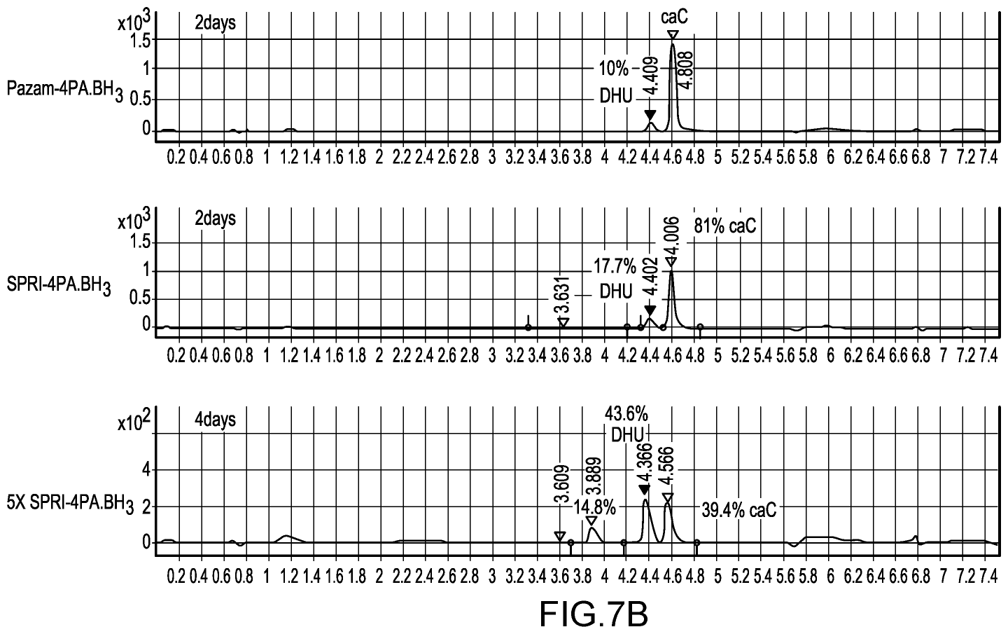

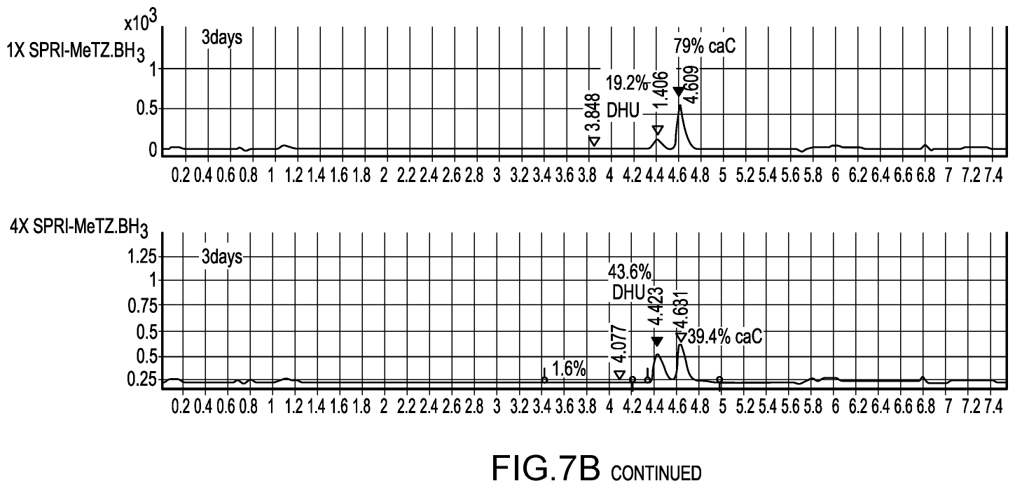

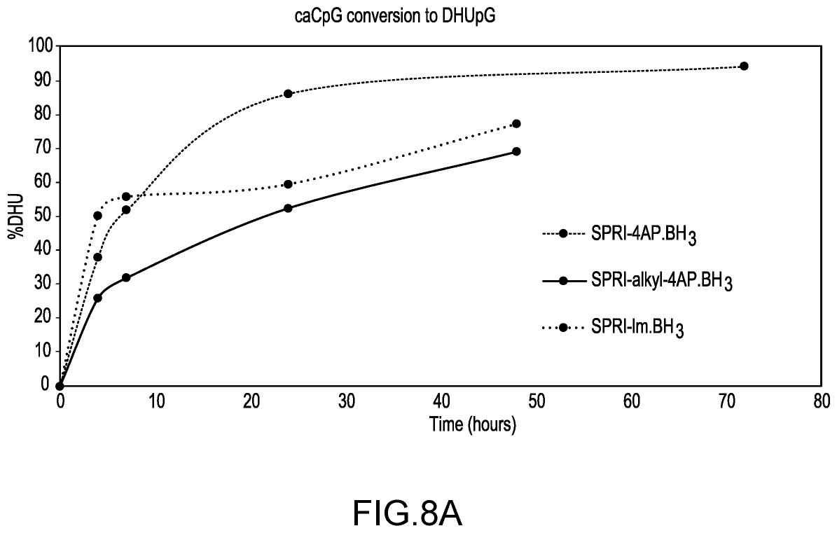

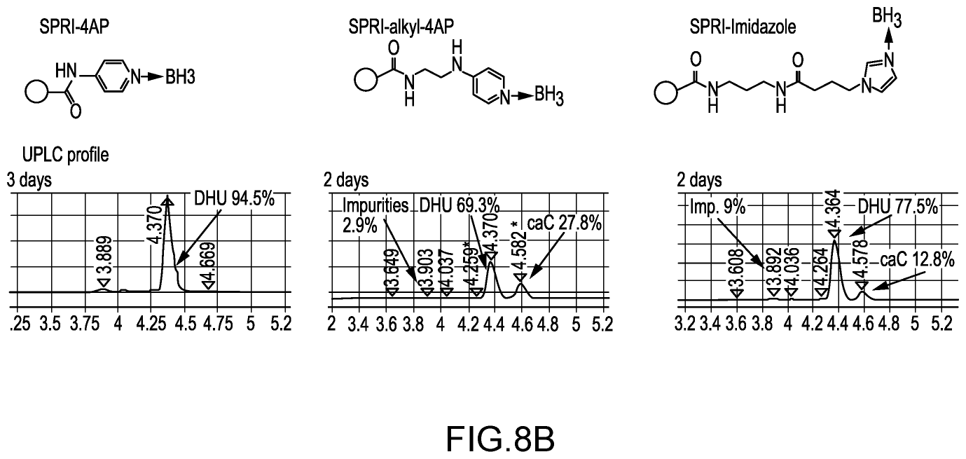

FIGS. 6A-6F schematically illustrate examples of possible coupling reactions in which SPRI beads or PAZAM beads are attached to both class A boranes and class B boranes. FIG. 6A schematically illustrates direct coupling of a 4-pyridylacetic acid borane complex (195) to PAZAM beads. The PAZAM beads, functionalized with an NH₂ group, were reacted with a 4-pyridylacetic acid borane complex (4PA·BH₃ (195)) in the presence of CD1 at room temperature overnight in DMF. This scheme resulted in the creation of a PAZAM-4PA·BH₃ bead/borane construct. FIGS. 6B-6F schematically illustrates various examples of amine-borane formation on beads using SPRI beads. FIG. 6B used the precursor (200) (4-N-(5-aminopentyl)-2-pyridin-4-ylacetamide) to form a SPRI-4PA·BH₃ bead/borane construct. FIG. 6C used the precursor (205) (4-N-(5-aminopentyl)-2-thiazol-4-ylacetamide) to form a SPRI-MeTZ·BH₃ bead/borane construct. FIG. 6D used 4-aminopyridine (210) to form a SPRI-4AP·BH₃ bead/borane construct. FIG. 6E used the precursor (215) 4-(2-aminoethyl)aminopyridine to form a SPRI-Alkyl-4AP·BH₃ bead/borane construct. FIG. 6F used an intermediate (220) as a precursor to form a SPRI-Im·BH₃ bead/borane construct. Some of these bead/borane constructs were tested as described in Examples 1˜4 and shown in FIGS. 7A-7B and FIGS. 8A-8B.

The coupling reactions illustrated in FIGS. 5A to 6F can be used to couple amine-boranes to beads with or without the use of a linker.

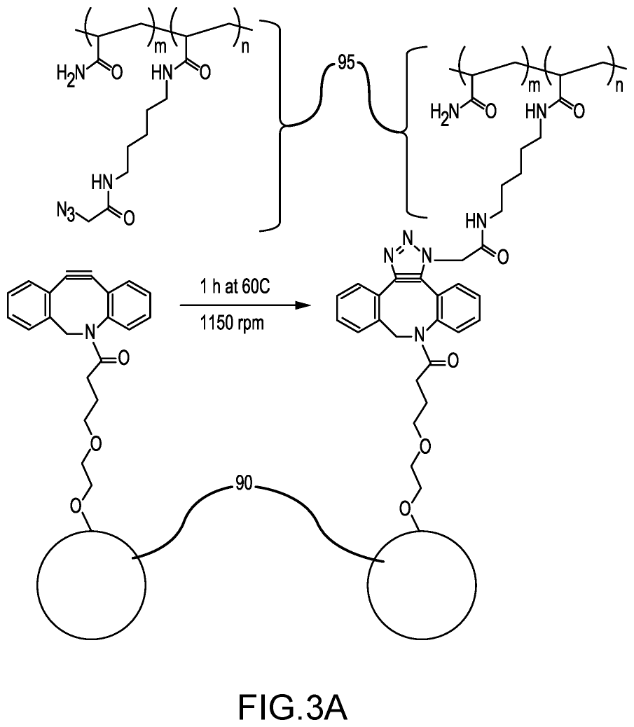

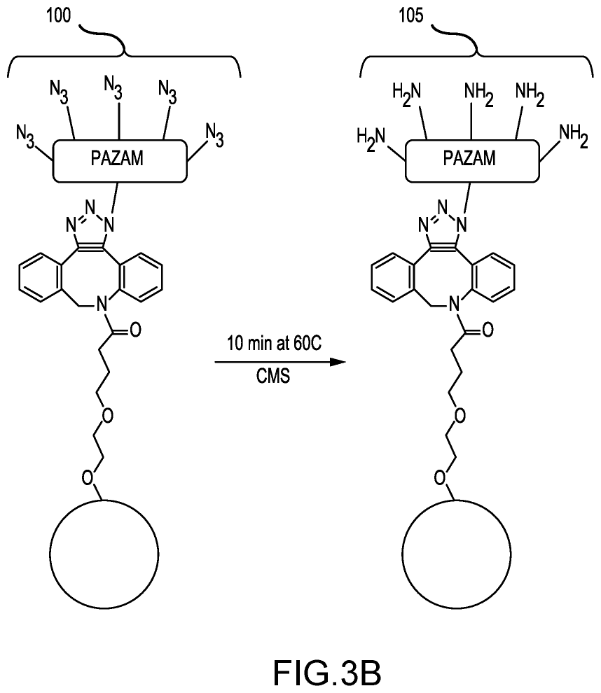

In some examples, the magnetic bead includes a SPRI bead. In some examples, the magnetic bead includes a PAZAM bead. FIGS. 3A-3B schematically illustrate coating DBCO (Dibenzocyclooctyne) functionalized magnetic beads with PAZAM in copper-free click reaction and conversion of azide into amine. FIG. 3A schematically illustrates an example of beads (90) coated with PAZAM. Magnetic beads of 1 um diameter were used at a concentration of 0.2 mg/ml and coated with an aqueous 0.5% (v/v) PAZAM solution. FIG. 3B schematically illustrates an example of converting azide groups on the PAZAM into amines. The azide groups (100) were converted into an amine (105) via a Staudinger reaction. A buffer solution that contained phosphines was used to convert the azide groups. In some examples, the magnetic bead includes a paramagnetic bead.

Some examples herein relate to a solid support coupled to an amine-borane. In some examples, the solid support includes any one or more of a bead, a microsphere, a filter, a surface of a tube or vessel, and a planar substrate. In some examples, the solid support is any solid support described herein. In some examples, the amine-borane is attached to the solid support through a covalent linkage between the amine-borane and the solid support. In some examples, the solid support includes an inert substrate or matrix, such as, for example, glass slides or polymer beads. In some examples, the solid support is magnetic. In some examples, the solid support is paramagnetic. In some examples, the amine-borane is immobilized on the solid support.

### Solid Supports

In the methods and compositions presented herein, amine-boranes can be immobilized to the solid support. In some examples, the amine-boranes are covalently immobilized to the support. When referring to immobilization of molecules (e.g., nucleic acids) to a solid support, the terms “immobilized” and “attached” are used interchangeably herein and both terms are intended to encompass direct or indirect, covalent or non-covalent attachment, unless indicated otherwise, either explicitly or by context. In certain examples covalent attachment may be preferred, but generally all that is required is that the molecules (e.g., amine-boranes) remain immobilized or attached to the support under the conditions in which it is intended to use the support, for example in applications requiring detecting methylation on dinucleotides and polynucleotides.

Certain examples may make use of solid supports made up of an inert substrate or matrix (e.g., glass slides, polymer beads etc.) which has been functionalized, for example by application of a layer or coating of an intermediate material comprising reactive groups which permit covalent attachment to biomolecules, such as polynucleotides. Examples of such supports include, but are not limited to, polyacrylamide hydrogels supported on an inert substrate such as glass, particularly polyacrylamide hydrogels as described in WO 2005/065814 and US 2008/0280773, the entire contents of each of which are incorporated by reference herein. In such examples, the biomolecules (e.g., polynucleotides) may be directly covalently attached to the intermediate material (e.g., the hydrogel) but the intermediate material may itself be non-covalently attached to the substrate or matrix (e.g., the glass substrate). The term “covalent attachment to a solid support” is to be interpreted accordingly as encompassing this type of arrangement.

Example covalent linkages include, for example, those that result from the use of click chemistry techniques. Example non-covalent linkages include, but are not limited to, non-specific interactions (e.g., hydrogen bonding, ionic bonding, van der Waals interactions etc.) or specific interactions (e.g., affinity interactions, receptor-ligand interactions, antibody-epitope interactions, avidin-biotin interactions, streptavidin-biotin interactions, lectin-carbohydrate interactions, etc.). Example linkages are set forth in U.S. Pat. Nos. 6,737,236; 7,259,258; 7,375,234 and 7,427,678; and US Pat. Pub. No. 2011/0059865 A1, the entire contents of each of which are incorporated by reference herein.

The terms “solid surface,” “solid support” and other grammatical equivalents herein refer to any material that is appropriate for or can be modified to be appropriate for the attachment of the amine-borane complexes. As will be appreciated by those in the art, the number of possible substrates is very large. Possible substrates include, but are not limited to, glass and modified or functionalized glass, plastics (including acrylics, polystyrene and copolymers of styrene and other materials, polypropylene, polyethylene, polybutylene, polyurethanes, Teflon™, etc.), polysaccharides, nylon or nitrocellulose, ceramics, resins, silica or silica-based materials including silicon and modified silicon, carbon, metals, inorganic glasses, plastics, optical fiber bundles, and a variety of other polymers. In some examples, particularly useful solid supports and solid surfaces are located within a flow cell apparatus. Example flow cells are set forth in further detail below.

In some examples, the solid support includes a patterned surface suitable for immobilization of amine-borane complexes in an ordered pattern. A “patterned surface” refers to an arrangement of different regions in or on an exposed layer of a solid support. For example, one or more of the regions can be features where one or more amine-boranes are present. The features can be separated by interstitial regions where amine-boranes complexes are not present. In some examples, the pattern can be an x-y format of features that are in rows and columns. In some examples, the pattern can be a repeating arrangement of features and/or interstitial regions. In some examples, the pattern can be a random arrangement of features and/or interstitial regions. In some examples, the amine-boranes complexes are randomly distributed upon the solid support. In some examples, the amine-borane complexes are distributed on a patterned surface. Example patterned surfaces that can be used in the methods and compositions set forth herein are described in U.S. Pat. No. 8,778,849 or U.S. Pat. No. 8,778,848, the entire contents of each of which are incorporated by reference herein.

In some examples, the solid support includes an array of wells or depressions in a surface. This may be fabricated as is generally known in the art using a variety of techniques, including, but not limited to, photolithography, stamping techniques, molding techniques and microetching techniques. As will be appreciated by those in the art, the technique used will depend on the composition and shape of the array substrate.

The composition and geometry of the solid support can vary with its use. In some examples, the solid support is a planar structure such as a slide, chip, microchip and/or array. As such, the surface of a substrate can be in the form of a planar layer. In some examples, the solid support includes one or more surfaces of a flowcell. The term “flowcell” as used herein refers to a chamber comprising a solid surface across which one or more fluid reagents can be flowed. Examples of flowcells and related fluidic systems and detection platforms that can be readily used in the methods of the present disclosure are described, for example, in Bentley et al., Nature 456:53-59 (2008), WO 04/018497; U.S. Pat. No. 7,057,026; WO 91/06678; WO 07/123744; U.S. Pat. Nos. 7,329,492; 7,211,414; 7,315,019; 7,405,281; and US 2008/0108082, the entire contents of each of which are incorporated by reference herein.

In some examples, the solid support or its surface is non-planar, such as the inner or outer surface of a tube or vessel. In some examples, the solid support includes microspheres or beads. By “microspheres” or “beads” or “particles” or grammatical equivalents herein is meant small discrete particles. Suitable bead compositions include, but are not limited to, plastics, ceramics, glass, polystyrene, methylstyrene, acrylic polymers, paramagnetic materials, thoria sol, carbon graphite, titanium dioxide, latex or cross-linked dextrans such as Sepharose, cellulose, nylon, cross-linked micelles and TEFLON, as well as any other materials outlined herein for solid supports may all be used. “Microsphere Detection Guide” from Bangs Laboratories, Fishers Ind. is a helpful guide. In certain examples, the microspheres are magnetic microspheres or beads.

The beads need not be spherical; irregular particles may be used. Alternatively or additionally, the beads may be porous. The bead sizes range from nanometers, i.e., 100 nm, to millimeters, i.e. 1 mm, with beads from about 500 nm to about 1 um being preferred, although in some examples smaller or larger beads may be used.

### WORKING EXAMPLES

### Example 1. Conversion of caC to DHU on Dinucleotides Using Class B Boranes Attached to Magnetic Beads

Class B boranes attached to magnetic beads do convert caCpG to DHUpG dinucleotides. The caCpG dinucleotide and the boranes were statically incubated at 40° C. in 50 ul of 500 mM of sodium acetate buffer, at a pH of 4.3. Different amounts of beads were used (see FIGS. 7A and 7B) with a 1× concentration being 0.2 mg per 50 uL reaction. The loading of amine borane onto SPRI beads was estimated at a maximum of 1 umol/mg of beads, leading to a maximum of 4 mM of amine borane in the reaction mixture for the 1× concentration. This concentration corresponded to eleven (11) equivalents of the dinucleotide.

FIG. 7A shows the kinetics of DHUpG formation from caCpG. FIG. 7B shows Ultra Performance Liquid Chromatography profiles after boranes attached to magnetic beads are incubated with dinucleotides for different time periods. Generally, the data show that the higher concentration of beads that were used, the higher percent of caC conversion to DHU. Although, the speed of conversion was shown to be much slower than when the borane was in solution, possibly due to the reduced accessibility of the borane.

### Example 2. Conversion of caC to DHU on Dinucleotides Using Class A Boranes Attached to Magnetic Beads

Class A boranes attached to magnetic beads do convert caCpG to DHUpG dinucleotides. The caCpG dinucleotide and the boranes were statically incubated at 40° C. in 50 ul of 500 mM of sodium acetate buffer, at a pH of 4.3 for up to 72 hours. A 1× concentration of beads was 0.2 mg per 50 uL reaction. The loading of amine borane onto SPRI beads was estimated at a maximum of 1 umol/mg of beads, leading to a maximum of 4 mM of amine borane in the reaction mixture for the 1× concentration. This concentration corresponded to eleven (11) equivalents of the dinucleotide. The formation of the DHUpG dinucleotide was followed by Ultra Performance Liquid Chromatography (UPLC), by injecting aliquots of the reactions at set time points.

FIG. 8A shows the kinetics of DHUpG formation from caCpG. FIG. 8B shows Ultra Performance Liquid Chromatography profiles showing the percent DHU formation using SPRI beads attached to different amine-boranes via different linkers. The data show that SPRI-4AP·BH₃ gave a full consumption of caCpG and resulted in ˜94.5% of DHUpG formation in less than three (3) days. Also, the data show that use of SPRI-alkyl-4AP·BH₃ resulted in a lower DHUpG conversion rate, which could be due to a lower loading of amine boranes onto the beads, or a lower amount of beads. Further, the data show that SPRI-Im·BH₃ seemed to be more active initially, but gave a lower final conversion. This could be due to a lower borane stability in the reaction conditions.

Comparison of the data in FIGS. 7A-7B to the data in FIGS. 8A-8B indicate that the class A boranes are much more reactive than the class B boranes.

### Example 3. Conversion of caC to DHU on a 7-Mer Using Class A Boranes Attached to Magnetic Beads

Class A boranes attached to magnetic beads do convert caC to DHU, on 7-mers. The 7-mer 5′-ATcaCGCTA-3′ and SPRI-4AP·BH₃ were statically incubated at 40° C. in 50 ul of 500 mM of sodium acetate buffer, at a pH of 4.3 for up to 48 hours. A 1× concentration of beads was 0.02 mg per 50 uL reaction. The loading of amine borane onto SPRI beads was estimated at a maximum of 1 umol/mg of beads, leading to a maximum of 0.4 mM of amine borane in the reaction mixture for the 1× concentration. The 7-mer oligonucleotide was used at a concentration of 40 uM, giving a ratio of 10 equivalent of borane to the 7 mer. The formation of the DHU-containing 7-mer 5′-ATDHUGCTA-3′ was followed by Ultra Performance Liquid Chromatography (UPLC), by injecting aliquots of the reaction at set time points.

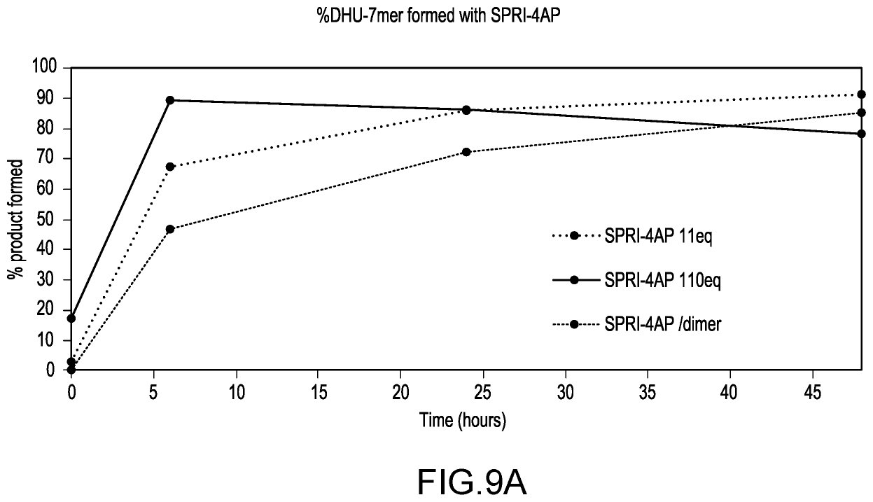

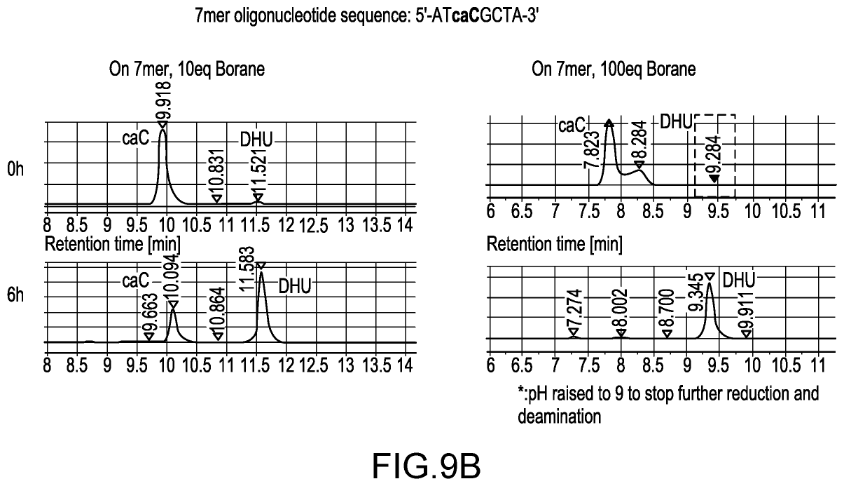

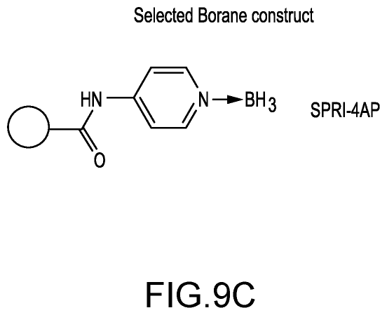

FIG. 9A shows the kinetics of percent DHU formation on a 7-mer in comparison to the dinucleotide caCpG. With 10 equivalents of SPRI-4AP·BH₃ (FIG. 9C) ˜85% of DHUpG dimer was reached in 48 hours. With 10 equivalents of SPRI-4AP·BH₃ construct (FIG. 9C) ˜90% of DHU 7-mer was reached in 48 hours (FIG. 9A). With 100 equivalents of the SPRI-4AP·BH₃ construct (FIG. 9C) ˜90% of DHU 7-mer was reached in only 5 hours (FIG. 9A). The data showed that at a high concentration of borane the conversion of caC to DHU on the 7 mer is fast, however a prolonged incubation time becomes detrimental reducing the final yield of conversion due to potential damage to the newly formed DHU 7 mer.

FIG. 9B shows Ultra Performance Liquid Chromatography profiles indicating DHU 7-mer formation, using the borane construct that is shown in FIG. 9C. DHU formation was tested using 10 equivalents of borane and 100 equivalents of borane. The data showed that a higher concentration of borane significantly improved the reaction kinetic.

### Example 4. Conversion of caC to DHU on a 20-Mer Using Class A Boranes Attached to Magnetic Beads

Class A boranes attached to magnetic beads do convert caC to DHU, on a 20-mer (5′-TTTCAGCTCcaCGGTCACGCTC-3′) (SEQ ID NO: 1). The 20-mer and the SPRI-4AP·BH₃ construct were statically incubated at 40° C. in 50 ul of 500 mM of sodium acetate buffer, at a pH of 4.3. A 1× concentration of beads was 0.1 mg per 50 uL reaction. The loading of amine borane onto SPRI beads was estimated at a maximum of 1 umol/mg of beads, leading to a maximum 2 mM of amine borane in the reaction mixture for the 1× concentration. The 20-mer oligo was used at a concentration of 20 uM, giving a ratio of 100 equivalent of borane to the 20-mer for the 1× concentration. The formation of the DHU-containing 20-mer (5′-TTTCAGCTCDHUGGTCACGCTC-3′) (SEQ ID NO: 2) was followed by Ultra Performance Liquid Chromatography (UPLC), by injecting aliquots of the reaction at set time points.

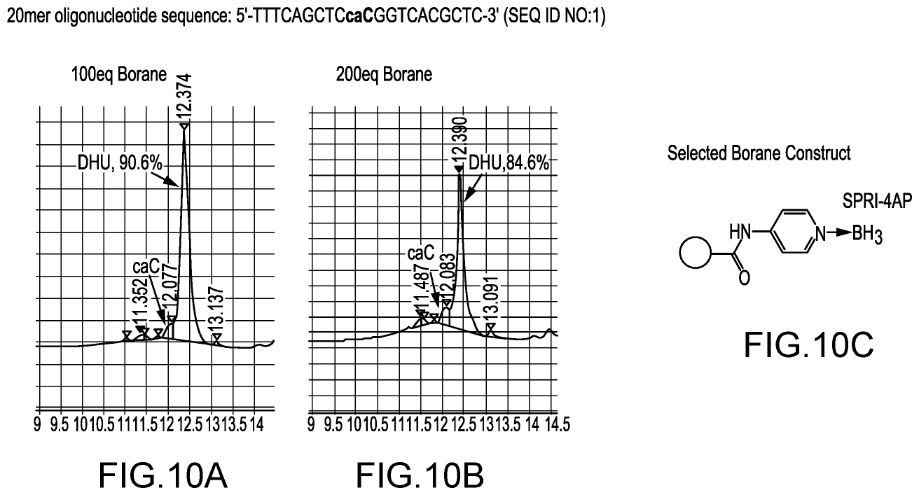

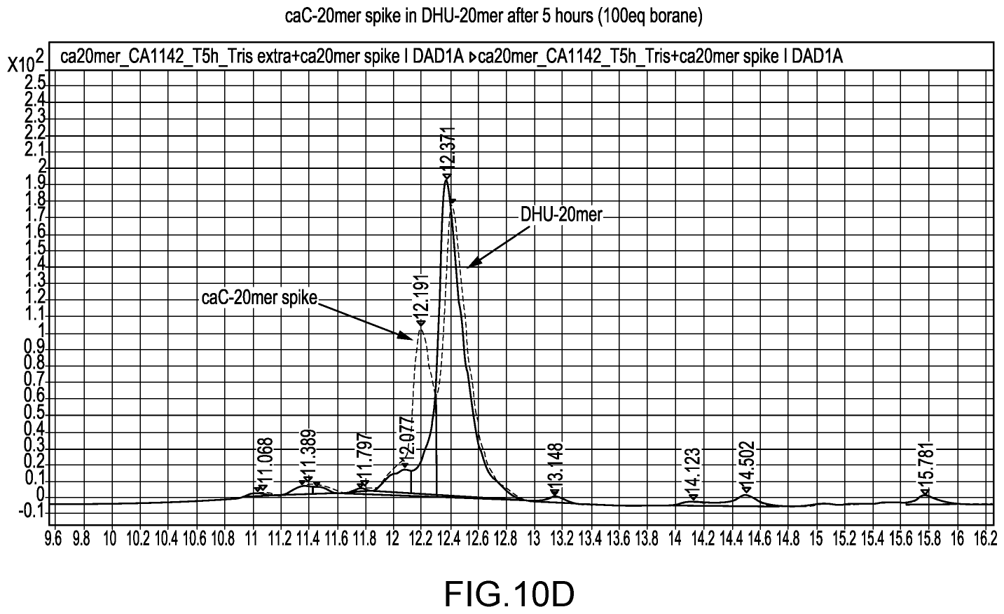

FIGS. 10A-10B and 10D show Ultra Performance Liquid Chromatography indicating formation of the DHU-containing 20-mer (5′-TTTCAGCTCDHUGGTCACGCTC-3′) (SEQ ID NO: 2) using the borane construct that is shown in FIG. 10C. In FIG. 10A, DHU formation was tested at 100 equivalents of borane. In FIG. 10B, DHU formation was tested at 200 equivalents of borane. FIG. 10D shows that same data as in FIG. 10A, with the superposition of a new UPLC trace showing the same reaction mixture spiked with the original caC-20 mer.

### Example 5. Coupling of a 4-Pyridylacetic Acid Borane Complex to PAZAM Beads

A 4-pyridylacetic acid borane complex was coupled to PAZAM beads as shown in FIG. 6A, using the protocol described below.

Sodium borohydride (1.5 eqv.) and powdered sodium bicarbonate (3 eqv.) were transferred to a 50 mL dry round bottom flask, charged with a magnetic stir-bar. 4-aminopyridine (5 mmol) was charged into the reaction flask followed by addition of reagent-grade tetrahydrofuran (1.9 mL per 5 mmol of 4apminopyridine) at room temperature. Under vigorous stirring, 1.9 mL of 14.4% v/v solution water in THF was added dropwise to prevent excessive frothing. After 16 hours, the reaction contents were filtered through sodium sulfate and celite and the solid residue washed with THF. Removal of the solvent in vacuo from the filtrate yielded the crude material, which was purified by flash chromatography (20% PET-EtOAc/35% PET-EtOAc) to lead to compound 195. ¹H NMR (400 MHZ, DMSO) δ 7.82 (d, J=7.1 Hz, 2H), 7.03 (s, 2H), 6.56 (d, J=7.2 Hz, 2H), 2.62-1.89 (br, 3H). ¹¹B NMR (128 MHz, DMSO) δ −12.94 (br).

PAZAM beads (0.2 mg) were placed in a 1 ml Eppendorf, place onto a magnet for 1 min and water removed. The beads were resuspended into 1 ml dry DMF, then placed onto a magnet for 1 minute. Supernatant was removed, and the same process was repeated 3 times. CDI (10 mg) was dissolved in 500 ul of dry DMF. Intermediate 195 (50 umoles) was added to the CDI solution and mix by vortexing. Finally, the mixture was transferred to the beads. The Eppendorf was sealed, the mixture vortexed, and then spun at room temperature in a rotisserie apparatus (17 rpm) for 17 hrs. the Eppendorf was placed onto a magnet for 1 minute, the supernatant was removed, and the beads were resuspended in DMF. The beads were placed onto a magnet for 1 minute and the supernatant removed. This process was repeated twice. The beads were finally resuspended in 200 ul of dry DMF as a stock solution.

### Example 6. Synthesis of a SPRI-4PA·BH₃ Bead/Borane Construct Using (4-N-(5-Aminopentyl)-2-Pyridin-4-Ylacetamide) as a Precursor

A SPRI-4PA-BH₃ bead/borane construct shown in FIG. 6B was synthesized using a (4-N-(5-aminopentyl)-2-pyridin-4-ylacetamide) as a precursor, through the protocol described below.

4-aminopyridine (500 mg, 1 eq) was dissolved in 6 ml of DCM. EDC (663 mg, 1.2 eq) and N-Boc-cadaverine (874 mg, 1.5 eq) was added. The reaction was stirred at room temperature overnight. The reaction was then diluted with DCM and washed 2 times with water, followed by 2 washes with phosphate buffer (pH=5). The aqueous phases were combined and further extracted twice with DCM. The organic phases were combined, dried with MgSO₄ and concentrated. The crude material was purified by flash chromatography (Silica gel, 100% DCM to 10% MeOH in DCM) to give a pure material (57%) (¹H NMR (400 MHZ, CDCl₃) δ 8.50 (dd, J=4.5, 1.6 Hz, 2H), 7.19 (dd, J=4.5, 1.6 Hz, 2H), 5.69 (s, 1H), 4.50 (s, 1H), 3.48 (s, 2H), 3.17 (dd, J=12.8, 7.0 Hz, 2H), 3.03 (dd, J=12.8, 7.0 Hz, 2H), 1.42 (m, 4H), 1.37 (s, 9H), 1.32-1.09 (m, 2H)).

This purified material was then diluted in 5 ml of DCM, and 1.3 ml of TFA was added slowly. The reaction was stirred at room temperature for 2 hours or until the deprotection was completed. The solvents were removed under vacuum. The crude material was purified by flash chromatography on a C18 column (gradient: 100% H₂O to 100% Acetonitrile) to lead to the intermediate 200 (¹H NMR (400 MHZ, DMSO) δ 8.69 (dd, J=4.9, 1.5 Hz, 2H), 8.26 (t, J=4 Hz, 1H), 7.72 (s, 1H), 7.64 (d, J=6.4 Hz, 2H), 3.65 (s, 2H), 3.06 (dd, J=12.7, 6.9 Hz, 2H), 2.76 (dd, J=15.0, 6.0 Hz, 2H), 1.62-1.47 (m, 2H), 1.46-1.39 (m, 2H), 1.36-1.19 (m, 2H)).

2 mg (2 ml, 1 eq) of SPRI beads (from a stock of beads suspended in water at 1 mg/ml) were placed into a 5 ml Eppendorf, then onto a magnet for 1 minute and the supernatant was removed. A solution of DMTMM (277 mg, 500 eq, in 1 ml of H₂O) was added to the beads and the beads were resuspended by vortexing. Compound 200 (221 mg, 500 eq), dissolved in 1 ml of H₂O, was then added to the bead mixture and vortexed. The Eppendorf was sealed and spun at room temperature on a rotisserie apparatus (17 rpm) for 17 hours. The bead mixture was placed onto a magnet for 1 minute, the supernatant was removed, and the beads were washed 3 times with H₂O, 2 times with DMF and 1 time with H₂O. The SPRI-4PA bead construct formed was resuspended in H₂O (2 ml) and kept in the refrigerator until needed.

1 mg of SPRI-4PA bead construct (1 mg, 1 eq) was placed onto a magnet for 1 minute and water removed. The beads were washed 3 times with dry THF. 200 ul of dry THF was added to the beads and the beads were well resuspended by vortexing. A 1M solution of THF·BH₃ complex in THF (2.5 ml, 2500 eq) was added to the bead suspension. The Eppendorf was sealed and the reaction was spun at room temperature on a rotisserie apparatus (17 rpm). The beads were placed onto a magnet for 1 minute and supernatant removed. The beads were washed 2 times with DMF and 3 times with H₂O. The final construct SPRI-4PA·BH₃ was resuspended in H₂O until needed and kept in the refrigerator.

### Example 7. Synthesis of SPRI-MeTZ·BH₃ Bead/Borane Construct Using (4-N-(5-Aminopentyl)-2-Thiazol-4-Ylacetamide) as a Precursor

A SPRI-MeTZ·BH₃ bead/borane construct shown in FIG. 6C was synthesized using a (4-N-(5-aminopentyl)-2-thiazol-4-ylacetamide), through the protocol described below.

2-(2-methylthiazol-4-yl) acetic acid (315 mg, 1 eq) was dissolved in 5 ml of DCM. EDC (461.5 mg, 1.2 eq) and N-Boc-cadaverine (607 mg, 1.5 eq) were added. The reaction was stirred at room temperature overnight. The reaction was then diluted with DCM and washed 2 times with water, followed by 2 washes with phosphate buffer (pH=5). The aqueous phases were combined and further extracted twice with DCM. The organic phases were combined, dried with MgSO₄ and concentrated. The crude material was purified by flash chromatography (Silicagel, 100% DCM to 10% MeOH in DCM) to give a pure material (96%) (¹H NMR (400 MHZ, CDCl₃) δ 6.88 (s, 1H), 6.73 (s, 1H), 4.47 (s, 1H), 3.59 (d, J=0.4 Hz, 2H), 3.17 (dd, J=12.9, 7.0 Hz, 2H), 3.02 (dd, J=13.0, 6.5 Hz, 2H), 2.66 (s, 3H), 1.48-1.33 (m, 13H), 1.29-1.15 (m, 2H)).

This purified material was then diluted in 5.6 ml of DCM, and 1.4 ml of TFA was added slowly. The reaction was stirred at room temperature for 2 h or until the deprotection was completed. The solvents were removed under vacuum. The crude material was purified by flash chromatography on a C18 column (gradient: 100% H₂O to 100% Acetonitrile) to lead to the intermediate 205 (¹H NMR (400 MHZ, DMSO) δ 7.98 (s, 1H), 7.70 (s, 2H), 7.17 (s, 1H), 3.50 (s, 2H), 3.06 (dd, J=12.7, 6.7 Hz, 2H), 2.76 (dd, J=13.3, 6.7 Hz, 2H), 2.61 (s, 3H), 1.57-1.49 (m, 2H), 1.46-1.39 (m, 2H), 1.33-1.24 (m, 2H)).

1 mg (1 ml, 1 eq) of SPRI beads (from a stock of beads suspended in water at 1 mg/ml) were placed into a 5 ml Eppendorf, then onto a magnet for 1 minute, and the supernatant was removed. A solution of DMTMM (138 mg, 500 eq, in 0.5 ml of H₂O) was added to the beads and the beads were resuspended by vortexing. Compound 205 (121 mg, 500 eq), dissolved in 0.5 ml of H₂O, was then added to the bead mixture and vortexed. The Eppendorf was sealed and spun at room temperature on a rotisserie apparatus (17 rpm) for 17 hours. The bead mixture was placed onto a magnet for 1 minute, the supernatant was removed, and the beads were washed 3 times with H₂O, 2 times with DMF and 1 time with H₂O. The SPRI-MeTZ bead construct formed was resuspended in H₂O (1 ml) and kept in the refrigerator until needed.

1 mg of SPRI-MeTZ bead construct (1 mg, 1 eq) was placed onto a magnet for 1 minute and water removed. The beads were washed 3 times with dry THF. 200 ul of dry THF was added to the beads and the beads were well resuspended by vortexing. A IM solution of THF·BH₃ complex in THF (2.5 ml, 2500 eq) was added to the bead suspension. The Eppendorf was sealed and the reaction was spun at room temperature on a rotisserie apparatus (17 rpm). The beads were placed onto a magnet for 1 minute and supernatant removed. The beads were washed 2 times with DMF and 3 times with H₂O. The final construct SPRI-MeTZ·BH₃ was resuspended in H₂O and kept in the refrigerator until needed.

### Example 8. Synthesis of a SPRI-4AP·BH₃ Bead/Borane Construct using Aminopyridine

A SPRI-4AP·BH₃ bead/borane construct shown in FIG. 6D was synthesized using aminopyridine, through the protocol described below.

2.5 mg (2.5 ml, 1 eq) of SPRI beads (from a stock of beads suspended in water at 1 mg/ml) were placed into a 5 ml Eppendorf, then onto a magnet for 1 minute and the supernatant was removed. The beads were washed 3 times with dry DMF. A solution of CDI (406 mg, 1000 eq, in 1 ml of DMF) was added to the beads and the beads were resuspended by vortexing. 4-aminopyridine (compound 210, 117.5 mg, 500 eq), dissolved in 0.5 ml of dry DMF, was then added to the bead mixture and vortexed. The Eppendorf was sealed and spun at room temperature on a rotisserie apparatus (17 rpm) for 17 hours. The bead mixture was placed onto a magnet for 1 minute, the supernatant was removed, and the beads were washed 3 times with DMF and 2 times with H₂O. The SPRI-4AP bead construct formed was resuspended in H₂O (2.5 ml) and kept in the refrigerator until needed.

1.8 mg of SPRI-4AP bead construct (1 eq) was placed onto a magnet for 1 min and water removed. The beads were washed 3 times with dry THF. 100 ul of dry THF was added to the beads and the beads were well resuspended by vortexing. A IM solution of THF·BH₃ complex in THF (4.5 ml, 2500 eq) was added to the bead suspension. The Eppendorf was sealed and the reaction was spun at room temperature on a rotisserie apparatus (17 rpm). The beads were placed onto a magnet for 1 minute and supernatant removed. The beads were washed 3 times with H₂O, 1 time with DMF and 1 time with H₂O. The final construct SPRI-4AP·BH₃ was resuspended in H₂O and kept in the refrigerator until needed.

### Example 9. Synthesis of a SPRI-Alkyl-4AP·BH₃ Bead/Borane Construct Using 4-(2-Aminoethyl)Aminopyridine as a Precursor

A SPRI-Alkyl-4AP·BH₃ bead/borane construct shown in FIG. 6E was synthesized using 4-(2-aminoethyl)aminopyridine as a precursor, through the protocol described below.

4-bromopyridine (2 g, 1 eq) was put in a flask under nitrogen atmosphere with a magnetic stirrer bar. Ethylenediamine (7 ml, 10 eq) was added to the flask and the reaction was heated to reflux, neat, for 1.5 hours or until reaction is finished (followed by LCMS). The reaction was cooled down and K₂CO₃ (5.7 g, 4 eq) was added to the mixture and the reaction was stirred at room temperature for 10 minutes. The solid was filtered, washed with 30 ml of toluene and then 100 ml of isoprona-2-ol. The filtrate was concentrated under vacuum. The crude material was then purified by flash chromatography (10 g Amino column, PE/EtOAc 0% to 100%, then EtOAc/MeOH 0% to 25%) to lead to the compound 215 (68%) (¹H NMR (400 MHZ, DMSO) δ 7.99 (dd, J=4.8, 1.5 Hz, 2H), 6.46 (dd, J=4.8, 1.6 Hz, 3H), 3.02 (dd, J=12.0, 6.3 Hz, 2H), 2.68 (t, J=6.4 Hz, 2H), 1.62 (d, J=74.8 Hz, 2H)).

2.5 mg (2.5 ml, 1 eq) of SPRI beads (from a stock of beads suspended in water at 1 mg/ml) were placed into a 5 ml Eppendorf, then onto a magnet for 1 minute and the supernatant was removed. A solution of DMTMM (346 mg, 500 eq, in 1 ml of H₂O) was added to the beads and the beads were resuspended by vortexing. Compound 215 (170 mg, 500 eq), dissolved in 0.5 ml of dry DMF, was then added to the bead mixture and vortexed. The Eppendorf was sealed and spun at room temperature on a rotisserie apparatus (17 rpm) for 17 hours. The bead mixture was placed onto a magnet for 1 min, the supernatant was removed, and the beads were washed 2 times with H₂O, 3 times with DMF and 2 times with H₂O. The SPRI-Alkyl-4AP bead construct formed was resuspended in H₂O (2 ml) and kept in the refrigerator until needed.

1.8 mg of SPRI-4AP bead construct (1 eq) was placed onto a magnet for 1 minute and water removed. The beads were washed 3 times with dry THF. 100 ul of dry THF was added to the beads and the beads were well resuspended by vortexing. A 1M solution of THF·BH₃ complex in THF (4.5 ml, 2500 eq) was added to the bead suspension. The Eppendorf was sealed and the reaction was spun at room temperature on a rotisserie apparatus (17 rpm). The beads were placed onto a magnet for 1 minute and supernatant removed. The beads were washed 3 times with H₂O, 1 time with DMF and 1 time with H₂O. The final construct SPRI-Alkyl-4AP·BH₃ was resuspended in H₂O and kept in the refrigerator until needed.

### Example 10. Synthesis of a SPRI-Im·BH₃ Bead/Borane Construct

A SPRI-Im·BH₃ bead/borane construct was synthesized through the protocol described below.

Synthesis of compound 220: Imidazole (2 g, 1 eq) was dissolved in 25 ml DCM and placed under nitrogen atmosphere. Add KOH (2.47 g, 1.5 eq), K₂CO₃ (4.06 g, 1 eq) and tetrabutylammonium bromide (190 mg, 0.02 eq). The reaction was stirred at room temperature for 30 min. Ethyl bromobutyrate (4.6 ml, 1.1 eq) was added dropwise under nitrogen atmosphere and stirred at reflux overnight. The formed precipitate was filtered and washed with DCM. The filtrate solution was washed with brine twice, dried with MgSO₄ and concentrated under vacuum. The crude material was then deprotected overnight with a 5M HCl solution (11.8 ml, 2 eq). The solvents were removed under vacuum. The crude was dissolved in H₂O and extracted 3 times with DCM. The aqueous phase was then concentrated under vacuum and purified by flash chromatography using a C18 column (120 g, 100% H₂O) to give compound 220 (¹H NMR (400 MHZ, D₂O) δ 8.66 (s, 1H), 7.40 (d, J=24.6 Hz, 2H), 4.19 (t, J=7.1 Hz, 2H), 2.30 (t, J=7.2 Hz, 2H), 2.08 (p, J=7.2 Hz, 2H)).

0.5 mg (0.5 ml, 1 eq) of SPRI beads (from a stock of beads suspended in water at 1 mg/ml) were placed into a 1.5 ml Eppendorf, then onto a magnet for 1 minute and the supernatant was removed. The beads were resuspended in 200 ul of H₂O. A solution of DMTMM (138 mg, 1000 eq, in 400 ul of H₂O) was added to the beads and the beads were resuspended by vortexing. Propylenediamine (41.7 ul, 1000 eq) was added to the bead mixture. The Eppendorf was sealed and spun on a rotisserie apparatus (17 rpm) overnight at room temperature. The bead mixture was placed onto a magnet for 1 minute, the supernatant was removed, and the beads were washed 3 times with H₂O, 2 times with DMF and 2 times with H₂O.

The intermediate construct (0.5 mg, 1 eq) was dissolved in 150 ul H₂O. A solution of DMTMM (69 mg, 1000 eq, in 300 ul of H₂O) was added to the beads and the beads were resuspended by vortexing. Compound 220 (39 mg, 900 eq), dissolved in 200 ul of H₂O, was then added to the bead mixture and vortexed. The Eppendorf was sealed and spun at room temperature on a rotisserie apparatus (17 rpm) for 17 hours. The bead mixture was placed onto a magnet for 1 min, the supernatant was removed, and the beads were washed 3 times with H₂O, 2 times with DMF and 2 times with H₂O. The SPRI-Im bead construct formed was resuspended in H₂O (0.5 ml) and kept in the refrigerator until needed.

0.25 mg of SPRI-Im bead construct (1 eq) was placed onto a magnet for 1 minute and water removed. The beads were washed 3 times with dry THF. 100 ul of dry THF was added to the beads and the beads were well resuspended by vortexing. A 1M solution of THF·BH₃ complex in THF (625 ul, 2500 eq) was added to the bead suspension. The Eppendorf was sealed and the reaction was spun at room temperature on a rotisserie apparatus (17 rpm). The beads were placed onto a magnet for 1 minute and supernatant removed. The beads were washed 3 times with H₂O, 1 time with DMF and 1 time with H₂O. The final construct SPRI-Im·BH₃ was resuspended in H₂O and kept in the refrigerator until needed.

### ADDITIONAL COMMENTS

While various illustrative examples are described above, it will be apparent to one skilled in the art that various changes and modifications may be made therein without departing from the disclosure. The appended claims are intended to cover all such changes and modifications that fall within the true spirit and scope of the examples provided herein.

It is to be understood that any respective features/examples of each of the aspects of the disclosure as described herein may be implemented together in any appropriate combination, and that any features/examples from any one or more of these aspects may be implemented together with any of the features of the other aspect(s) as described herein in any appropriate combination to achieve the benefits as described herein.

1. A method comprising:
oxidizing any 5-methylcytosine (5-mC) or 5-hydroxymethylcytosine (5-hmC) on a polynucleotide to 5-carboxylcytosine (5-caC) or 5-formylcytosine (5-fC);
reducing the 5-caC or 5-fC to 5,6-dihydrouracil (DHU) using an amine-borane attached to a solid support; and
detecting the 5-methylcytosine or 5-hydroxymethylcytosine using the DHU.

2. The method of claim 1, wherein the solid support comprises any one or more of a bead, a microsphere, a filter, a surface of a tube or vessel, and a planar substrate.

3. The method of claim 2, wherein the bead comprises a magnetic bead.

4. (canceled)

5. The method of claim 2, wherein the microsphere comprises a magnetic microsphere.

6. The method of claim 2, wherein the bead comprises a solid-phase reversible immobilization (SPRI) bead.

7. The method of claim 1, wherein a ten-eleven translocation (TET) dioxygenase is used to oxidize any 5-mC or 5-hmC.

8. The method of claim 1, wherein oxidizing any 5-mC or 5-hmC comprises contacting 5-mC or 5-hmC with one or more chemical reagents.

9. The method of claim 1, further comprising contacting the solid support with the 5-caC or 5-fC.

10. (canceled)

11. The method of claim 1, further comprising using the solid support to separate the DHU from the amine-borane and any other reaction products.

12. (canceled)

13. The method of claim 1, wherein the oxidizing step and reducing step take place in a solution.

14. The method of claim 13, further comprising absorbing the polynucleotide onto the solid support.

15. The method of claim 14, further comprising eluting the polynucleotide such that it is released from the solid support.

16. The method of claim 13, wherein the polynucleotide remains in the solution.

17. The method of claim 1, wherein the solid support is attached to a linker and the amine-borane attaches to the solid support through the linker.

18. A composition, comprising:
a magnetic bead coupled to an amine-borane.

19. The composition of claim 18, wherein the composition further comprises a linker that couples the magnetic bead to the amine-borane.

20. The composition of claim 18, wherein the magnetic bead comprises a solid-phase reversible immobilization (SPRI) bead.

21. (canceled)

22. The composition of claim 18, wherein the magnetic bead is connected to a first functional group (F₁) and the linker is connected to a second functional group (F₂) and to a third functional group (F₃), wherein the linker couples the magnetic bead to the amine-borane via coupling F₁ to F₂, and coupling F₃ to the amine-borane.

23. A method, comprising:
coupling a magnetic bead to a linker to create a composite magnetic bead-linker structure; and
coupling the composite magnetic bead-linker structure to an amine-borane.

24. The method of claim 23, wherein the magnetic bead is connected to a first functional group (F₁) and the linker is connected to a second functional group (F₂) and to a third functional group (F₃), and wherein the magnetic bead is coupled to the linker via coupling F₁ to F₂ to form the composite magnetic bead-linker structure, and wherein the composite magnetic bead-linker structure is coupled to the amine-borane via coupling F₃ to the amine-borane.


SEQUENCE LISTING (5 sequences)

SEQ ID NO: 1 - DNA, 20 bp, synthetic construct
  source 1..20: mol_type = other DNA; note = 20-mer oligonucleotide
  modified_base 10: mod_base = OTHER; note = carboxylcytosine
tttcagctccggtcacgctc

SEQ ID NO: 2 - DNA, 20 bp, synthetic construct
  source 1..20: mol_type = other DNA; note = DHU-containing 20-mer
  modified_base 10: mod_base = OTHER; note = dihyrouracil
tttcagctctggtcacgctc

SEQ ID NO: 3 - DNA, 12 bp, synthetic construct
  source 1..12: mol_type = other DNA; note = polynucleotide
  modified_base 10: mod_base = OTHER; note = methylcytosine
ccgtcggaccgc

SEQ ID NO: 4 - DNA, 12 bp, synthetic construct
  source 1..12: mol_type = other DNA; note = polynucleotide
  modified_base 10: mod_base = OTHER; note = carboxylcytosine
cctgcggaccgc

SEQ ID NO: 5 - DNA, 12 bp, synthetic construct
  source 1..12: mol_type = other DNA; note = polynucleotide
  modified_base 10: mod_base = OTHER; note = dihydrouracil
ccgtcggactgc